(19) United States
(12) Patent Application Publication
(10) Pub. No.: US 2014/0275558 A1
(43) Pub. Date: Sep. 18, 2014

(54) PREPARATION OF HALOALKOXYARYLHYDRAZINES AND INTERMEDIATES THEREFROM

(71) Applicant: Dow AgroSciences LLC, Indianapolis, IN (US)

(72) Inventors: Ronald ROSS, JR., Zionsville, IN (US); Peter BORROMEO, Fishers, IN (US)

(73) Assignee: Dow AgroSciences LLC, Indianapolis, IN (US)

(21) Appl. No.: 14/192,421

(22) Filed: Feb. 27, 2014

Related U.S. Application Data
(60) Provisional application No. 61/778,486, filed on Mar. 13, 2013.

(51) Int. Cl. C07D 249/08; C07C 251/86; C07C 241/02

(52) U.S. Cl. CPC C07D 249/08; C07C 241/02; C07C 251/86
USPC 548/265.8; 564/314; 564/251

(57) ABSTRACT

This document is related to the field of preparation of haloalkoxyarylhydrazines and certain intermediates derived therefrom, where said intermediates are useful in the preparation of certain pesticides disclosed in U.S. Pat. No. 8,178,658.

### CROSS REFERENCE TO RELATED APPLICATIONS

This application claims the benefit of U.S. Provisional Patent Application Ser. No. 61/778,486 filed Mar. 13, 2013, the entire disclosure of which is hereby expressly incorporated by reference.

### FIELD OF THE DISCLOSURE

This document is related to the field of preparation of haloalkoxyarylhydrazines and intermediates therefrom, where said intermediates are useful in the preparation of certain pesticides.

### BACKGROUND OF THE DISCLOSURE

U.S. Pat. No. 8,178,658 discloses pesticidal compositions comprising a compound having the following structure:

[chemical structure]

wherein Ar₁, Het, Ar₂, J, L, K, Q, R1, R2, R3, and R4 are disclosed in the patent. While processes are disclosed on how to make such compounds, and such processes are useful, it is desired to have more useful processes to make these compounds. In particular, it is desirable to have more commercially useful routes to certain substituted triaryl intermediates disclosed in the patent that are useful in producing the compounds of Formula A-1.

### DESCRIPTION OF THE DISCLOSURE

Throughout this document, all temperatures are given in degrees Celsius, and all percentages are weight percentages unless otherwise stated.

The term “alkyl”, as well as derivative terms such as “haloalkoxy”, as used herein, include within their scope straight chain, branched chain and cyclic moieties. Thus, typical alkyl groups are methyl, ethyl, propyl, butyl, pentyl, hexyl, 1-methylethyl, 1,1-dimethylethyl, 1-methylpropyl, 2-methylpropyl, cyclopropyl, cyclobutyl, cyclopentyl and cyclohexyl. The term “haloalkoxy” includes alkoxy groups substituted with from one to the maximum possible number of halogen atoms, all combinations of halogens included. Unless specifically defined otherwise, the term “halogen” or “halo” includes fluorine, chlorine, bromine and iodine.

[chemical structure]

Haloalkoxyarylhydrazine salts ((HX)ₙ where n=0, 1, or 2) of Formula 1.3, wherein R is a (C₁-C₆)haloalkoxy, for example, trifluoromethoxy and pentafluoroethoxy, can be prepared as illustrated in Scheme 1. In general, in step a, a haloalkoxyarylhalide of Formula 1 (wherein X is F, Cl, Br, or I, preferably Br) is reacted with a hydrazone of Formula 1.1 to give an intermediate haloalkoxyarylhydrazone of Formula 1.2; wherein R₁ and R₂ are independently aryl or heteroaryl, for example, phenyl, pyridyl, napthyl, and thienyl, substituted aryl or heteroaryl (wherein said substituents do not adversely affect the reaction, and may be halo, alkyl, alkoxy, haloalkyl, and nitro), or tertiary alkyl, for example, C(CH₃)₃, C(CH₃)₂C(CH₃)₂, and C(CH₃)₂C(CH₃)₃. In step b, the intermediate hydrazone of Formula 1.2 is hydrolyzed to form said haloalkoxyarylhydrazine salts ((HX)ₙ where n=0, 1, or 2) of Formula 1.3.

Step a is conducted in a preferably anhydrous, oxygen-free, aromatic solvent, for example, benzene, toluene, xylenes, or mixtures thereof. A haloalkoxyarylhalide of Formula 1 is reacted with a hydrazone of Formula 1.1 in the presence of a palladium catalyst complex comprising palladium and a ligand, for example, tetrakis(triphenyl-phosphine)palladium(0) (Pd(PPh₃)₄) and tris(dibenzylideneacetone)dipalladium(0) (Pd₂(dba)₃). Palladium catalyst complexes can be made by known methods in the art, for example, the aforementioned palladium catalyst complexes can be prepared as in D. R. Coulson et al. Inorg. Synth. 1972, 13, 121, and L. Paquette, Encyclopedia of Reagents for Organic Synthesis 1996, Ed.: J. Wiley and Sons: Sussex, England, respectively; in general they can be made by reacting, for example:

(a) palladium (II) acetate (Pd(OAc)₂);

(b) palladium (II) chloride (PdCl₂);

(c) sodium tetrachloropalladate (Na₂PdCl₄); and/or

(d) bis(dibenzylideneacetone) palladium(II) (Pd(dba)₂),

with a ligand, for example:

(a) triphenylphosphine (PPh₃);

(b) 1,1′-bis(diphenylphosphino)ferrocene (dppf);

(c) dibenzylideneacetone (dba);

(d) 2′-bis(diphenylphosphino)-1,1′-binaphthyl (BINAP); and/or

(e) 2-dicyclohexylphosphino-2′-methylbiphenyl (MePhos).

Approximately a 1:1 molar ratio of the haloalkoxyarylhalide of Formula 1 and a hydrazone of Formula 1.1 may be used, however, molar ratios of about 1.5:1 to about 1:1.5 may also be used. The reaction of the haloalkoxyarylhalide of Formula 1 with the hydrazine of Formula 1.1 is conducted in the presence of an organic base, for example, sodium tert-butoxide (NaO^{t}Bu), potassium tert-butoxide (KO^{t}Bu), and lithium hexamethyldisilazide (LiHMDS), an inorganic base, for example, sodium carbonate (Na₂CO₃), potassium carbonate (K₂CO₃), cesium carbonate (Cs₂CO₃), and tripotassium phosphate (K₃PO₄), or mixtures thereof. The reaction is conducted at a pH from about 8 to about 14 and preferably from about 12 to about 14. The reaction is conducted at a temperature from about 20° C. to about 100° C. and preferably from about 95° C. to about 100° C. The reaction is conducted at about atmospheric pressure, however, higher or lower pressures can be used.

The hydrolysis conducted in step b is conducted in a polar, protic solvent, such as an alcohol, for example, methanol (MeOH) and ethanol (EtOH), in the presence of an aqueous inorganic acid, for example, hydrochloric acid (HCl), nitric acid (HNO₃), phosphoric acid (H₃PO₄), sulphuric acid (H₂SO₄), hydrofluoric acid (HF), hydrobromic acid (HBr), perchloric acid (HClO₄), tetrafluoroboric acid (HBF₄), or mixtures thereof. The reaction is conducted at a pH from about −1 to about 4 and preferably from about −1 to about 1. The reaction is conducted at a temperature from about 65° C. to about 85° C. and preferably from about 75° C. to about 80° C.

Intermediate haloalkoxyarylhydrazone of Formula 1.2 that are particularly preferred are

[chemical structure]

[chemical structure]

Arylalkoxyimidate salts of Formula 2.2, wherein R₁ is NO₂, C(═O)OH or a (C₁-C₆) ester thereof (C(═O)O(C₁-C₆)alkyl), for example, methyl (C(═O)OCH₃) or ethyl ester (C(═O)OCH₂CH₃), can be prepared as outlined in Scheme 2. In step a, benzonitriles of Formula 2.1 are reacted with anhydrous inorganic acids in an alcohol to produce said arylalkoxyimidate salts, wherein R₂ is (C₁-C₆)alkyl.

In Step a benzonitriles of Formula 2.1 are treated with an anhydrous inorganic acid (HX, wherein X is F, Cl, Br, or I, preferably Cl or Br), for example, HCl or HBr in a polar protic solvent, for example, an alcohol (R₂OH), for example, MeOH, EtOH, n-butanol, isopropanol, or mixtures thereof. In some embodiments, HX gas is introduced directly into a solution of the benzonitrile of Formula 2.1 in R₂OH via a sparge tube. The reaction is conducted at a temperature from about −10° C. to about −5° C. and preferably from about 0° C. to about −5° C. during the HX sparge. It is preferred if the temperature is raised to about 25° C. following the addition of the HX. HX gas may be introduced into the reaction system at pressures ranging from about atmospheric pressure to about 3500 kPa. Alternatively, solutions of benzonitriles of Formula 2.1, in a variety of organic solvents, for example, tetrahydrofuran (THF), ethyl acetate (EtOAc), dichloromethane (CH₂Cl₂), toluene, or mixtures thereof, are treated with an anhydrous inorganic acid (HX), for example, HCl or HBr, in the presence of an alcohol (R₂OH). Molar ratios of benzonitriles of Formula 2.1 to the alcohol are from about 1:1 to about 1:10, however, molar ratios of about 1:1000 to about 1000:1 may also be used. In another embodiment, HX is generated in situ via the decomposition of an acyl halide, such as, for example, acetyl chloride and acetyl bromide, when said acyl halide is contacted with R₂OH. In another embodiment thionyl chloride is used as a source of HCl. In this method the acyl halide may be added to a solution of the benzonitrile of Formula 2.1 in R₂OH or may be added to the R₂OH first, followed by the addition of the benzonitrile of Formula 2.1 to the pre-formed solution of HX. In both cases, the reaction is conducted at a temperature from about −10° C. to about −5° C. and preferably from about 0° C. to about−5° C. during the HX formation, and preferably the temperature is raised to about 25° C. following the addition.

In some embodiments, subjecting benzonitriles of Formula 2.1, wherein R₁ is nitro or a benzoate ester, to one of the described methods affords alkoxyimidate salts of Formula 2.2, wherein R₁ is as defined and R₂ is derived from R₂OH. In another embodiment, subjecting benzonitriles of Formula 2.1, wherein R₁ is a carboxylic acid, to one of the described methods affords alkoxyimidate salts of Formula 2.2, wherein R₁ is a mixture of the carboxylic acid and ester, wherein the R₁ ester and R₂ are both derived from R₂OH, e.g., when R₂OH is MeOH, R₁ is the methyl ester and R₂ is a methyl group.

[chemical structure]

1,3-diaryltriazoles of Formula 3.2 can be prepared as illustrated in Scheme 3. In step a, haloalkoxyarylhydrazine of Formula 1.3 is reacted with arylalkoxyimidate of Formula 2.2 to produce an intermediate iminohydrazine of Formula 3.1. In step b, the iminohydrazine is cyclized using a formate source, such as, for example, formic acid, formate esters, such as methyl- and ethyl formate, and orthoesters, such as, trimethyl- and triethyl orthoformate, to afford said 1,3-diaryltriazoles of Formula 3.2. Optionally, haloalkoxyarylhydrazine of Formula 1.3 and arylalkoxyimidate of Formula 2.2 can be in the form of free bases.

In step a, solutions of arylalkoxyimidate salts of Formula 2.2 in a weakly alkaline, heterocyclic solvent, such as pyridine, lutidine, or mixtures thereof, or in a non-basic, polar, aprotic solvent such as, for example, acetonitrile (MeCN) and THF, in the presence of organic or inorganic bases are reacted with haloalkoxyarylhydrazine salts of Formula 1.3 to produce an intermediate iminohydrazine of Formula 3.1. Suitable examples of organic and inorganic bases are pyridine, trialkylamines, such as, trimethylamine, triethylamine (TEA), and diisopropylethylamine (DIPEA), and alkali carbonates, such as, Na₂CO₃ and K₂CO₃, respectively. The reaction is conducted at a temperature from about −10° C. to about 10° C. and preferably from about 0° C. to about −5° C. during the addition of the hydrazine, and then the temperature is preferably raised to about 25° C. following the addition.

In step b, the intermediate iminohydrazine of Formula 3.1 is cyclized using a formate source. The reaction is conducted at a temperature from about 20° C. to about 100° C. and preferably from about 95° C. to about 100° C., which effectively enables cyclization to form the 1,3-diaryltriazole of Formula 3.2.

[chemical structure]

1,3-diaryl triazole compounds of Formula 4.2 and Formula 4.3 can be prepared according to Scheme 4. In method a, intermediate 1,3-diaryltriazoles of Formula 3.2, wherein R₁ is an ester, can be saponified to give 1,3-diaryltriazoles substituted with a carboxylic acid of Formula 4.2. In method b, intermediate 1,3-diaryltriazole of Formula 3.2), wherein R₁ is nitro, can be reduced to give 1,3-diaryltriazoles substituted with an amine of Formula 4.3.

Method a can be conducted in a polar, protic solvent, such as an alcohol, for example, MeOH, EtOH, n-butanol, isopropanol, or mixtures thereof, or in a polar, aprotic solvent such as THF, in the presence of an alkali hydroxide base, for example, sodium (NaOH), potassium (KOH), or lithium hydroxide (LiOH), and water. The reaction can be conducted at a temperature from about 20° C. to about 60° C. and preferably from about 20° C. to about 30° C. The pH of the reaction mixture is from about 8 to about 14 and preferably from about 10 to about 12.

Method b can be carried out in a wide variety of organic solvents including, for example, polar, protic solvents, such as alcohols, e.g., MeOH, EtOH, n-butanol, isopropanol, or mixtures thereof, polar, aprotic solvents, such as THF and EtOAc, or organic acids, for example, acetic acid, in the presence of a catalyst, such as palladium on carbon or palladium hydroxide on carbon, preferably palladium hydroxide on carbon (10%), and a hydrogen source, for example hydrogen gas, ammonium salts, e.g., ammonium formate, and cyclohexadiene. The reaction can be conducted at a temperature from about 20° C. to about 50° C. and preferably from about 20° C. to about 30° C. The reaction can be conducted at a pressure from about 101 kPa to about 689 kPa and preferably from about 101 to about 345 kPa. See also WO 2009/102736 A1.

1,3-diaryltriazole of Formula 4.2 and Formula 4.3 can be used as intermediates to form pesticides disclosed in U.S. Pat. No. 8,178,658 as disclosed therein.

### EXAMPLES

These examples are for illustration purposes and are not to be construed as limiting the disclosure to only the embodiments disclosed in these examples.

Starting materials, reagents, and solvents that were obtained from commercial sources were used without further purification. Anhydrous solvents were purchased as Sure/Seal™ from Aldrich and were used as received. Melting points were obtained on a Thomas Hoover Unimelt capillary melting point apparatus or an OptiMelt Automated Melting Point System from Stanford Research Systems and are uncorrected. Examples using “room temperature” were conducted in climate controlled laboratories with temperatures ranging from about 20° C. to about 24° C. Molecules are given their known names, named according to naming programs within ISIS Draw, ChemDraw or ACD Name Pro. If such programs are unable to name a molecule, the molecule is named using conventional naming rules. ¹H NMR spectral data are in ppm (5) and were recorded at 300, 400 or 600 MHz, and ¹³C NMR spectral data are in ppm (8) and were recorded at 75, 100 or 150 MHz, unless otherwise stated.

### Example 1

### Preparation of (4-(perfluoroethoxy)phenyl)hydrazine hydrochloride

[chemical structure]

### Step 1. Preparation of 1-(diphenylmethylene)-2-(4-(perfluoroethoxy)phenyl)-hydrazine

To a dry 2 L round bottomed flask fitted with an overhead mechanical stirrer, nitrogen inlet, thermometer, and reflux condenser were added 1 bromo-4-(perfluoroethoxy)-benzene (100 g, 344 mmol), benzophenone hydrazone (74.2 g, 378 mmol), and BINAP (4.28 g, 6.87 mmol), and the mixture was suspended in anhydrous toluene (500 mL). To exclude oxygen, argon was sparged into the mixture for ten minutes (min) prior to and during the addition of Pd(OAc)₂ (1.54 g, 6.87 mmol) and NaO^{t}Bu (49.5 g, 515 mmol), which was added in portions. The argon sparge was halted and the brown mixture was warmed to 100° C. and stirred for 3 hours (h). The reaction was cooled to room temperature and poured into water (500 mL) and the aqueous mixture was extracted with EtOAc (3×200 mL). The combined organic extracts were washed with water, washed with saturated aqueous sodium chloride (NaCl), dried over anhydrous magnesium sulfate (MgSO₄), filtered, and concentrated under reduced pressure on a rotary evaporator. The crude product was purified by flash column chromatography using 0-100% (v/v) EtOAc/hexanes as eluent to give the title compound as a red oil (123.3 g, 88%): ¹H NMR (400 MHz, CDCl₃) δ δ 7.63-7.56 (m, 4H), 7.55 (t, J=1.5 Hz, 1H), 7.51 (d, J=4.7 Hz, 1H), 7.36-7.26 (m, 5H), 7.13-7.04 (m, 4H); ¹⁹F NMR (376 MHz, CDCl₃) δ −85.94, −87.84; ¹³C NMR (101 MHz, CDCl₃) δ 145.23, 143.46, 141.24, 138.06, 132.53, 129.74, 129.41, 129.03, 128.30, 128.23, 126.57, 122.82, 113.45.

### Step 2. Preparation of (4-(perfluoroethoxy)phenyl)hydrazine hydrochloride

To a dry 250 mL round bottomed flask equipped with a magnetic stir bar, thermometer, and reflux condenser were added 1-(diphenylmethylene)-2-(4-(perfluoroethoxy)phenyl)hydrazine (63.6 g, 157 mmol), EtOH (50 mL), and concentrated HCl (100 mL, about 1.20 mol), and the reaction was warmed to 85° C. and stirred for 5 h. The reaction was cooled to room temperature and the dark slurry was concentrated to a brown paste on a rotary evaporator. The paste was slurried in CH₂Cl₂ (200 mL) and the resulting solid was collected by vacuum filtration and dried under vacuum at 40° C. to give the title compound as a tan solid (36.0 g, 82%): ¹H NMR (400 MHz, DMSO-d₆) δ 10.47 (s, 3H), 8.62 (s, 1H), 7.43-7.18 (m, 2H), 7.20-6.93 (m, 2H); ¹⁹F NMR (376 MHz, DMSO-d₆) 5-85.30, ×87.02; ESIMS m/z 243.15 ([M+H]⁺).

### Example 2

### Preparation of (diphenylmethylene)hydrazine-¹⁵N₂

[chemical structure]

To a 250 mL round bottom flask equipped with a magnetic stir bar, temperature probe, and reflux condenser were added benzophenone (9.0 g, 49.4 mmol), ethylene glycol (90 mL) and hydrazine monohydrate-¹⁵N₂ (3.86 g, 74.1 mmol), and the mixture was warmed to 100° C. The nearly homogeneous solution was stirred at 100° C. for 15 h and cooled to room temperature. The resulting solid was collected by vacuum filtration, dissolved in EtOAc (80 mL), and washed with water (5×50 mL). The organic phase was dried over MgSO₄, filtered, and concentrated to a white/slightly yellow solid. The solid was dried under vacuum to give the title compound as a slightly yellow solid (7.26 g, 70%): mp 96-98° C.; ¹H NMR (400 MHz, DMSO-d₆) δ 7.57 (t, J=7.4 Hz, 2H), 7.48 (dd, J=10.6, 4.2 Hz, 1H), 7.38-7.18 (m, 7H), 6.34 (s, 1H), 6.14 (s, 1H); ESIMS m/z 199 ([M+H]⁺).

### Example 3

### Preparation of (4-(perfluoroethoxy)phenyl)hydrazine hydrochloride-¹⁵N₂

[chemical structure]

### Step 1. Preparation of 1-(diphenylmethylene)-2-(4-(perfluoroethoxy)phenyl)-hydrazine-¹⁵N₂

To a dry 250 mL round bottomed flask equipped with a magnetic stir bar, nitrogen inlet, and reflux condenser were added 1 bromo-4-(perfluoroethoxy)benzene (9.69 g, 33.3 mmol), benzophenone hydrazone-¹⁵N₂ (7.26 g, 36.6 mmol), and BINAP (0.415 g, 0.67 mmol), and the mixture was suspended in anhydrous toluene (35 mL). To exclude oxygen, nitrogen was sparged into the mixture for 30 min prior to and during the addition of Pd(OAc)₂ (0.149 g, 0.67 mmol) and NaO^{t}Bu (4.80 g, 49.9 mmol), which was added in portions. The nitrogen sparge was halted and the brown mixture was warmed to 100° C. and stirred for 3 h. The reaction mixture was cooled to room temperature and stirred for 16 h, poured into water (35 mL), and the resulting aqueous mixture was extracted with EtOAc (3×35 mL). The combined organic extracts were washed with water, washed with brine, dried over MgSO₄, filtered, and concentrated under reduced pressure on a rotary evaporator. The crude product was purified by flash column chromatography using 0-10% EtOAc/hexanes as eluent to give the title compound as a yellow oil (8.98 g, 65%): ¹H NMR (400 MHz, DMSO-d₆) δ 9.14 (dd, J=94.2, 2.3 Hz, 1H), 7.58 (dtt, J=8.8, 5.7, 1.5 Hz, 3H), 7.45 (dd, J=8.2, 1.3 Hz, 2H), 7.38-7.26 (m, 7H), 7.23-7.14 (m, 2H); ¹⁹F NMR (376 MHz, DMSO-d₆) δ-85.30, −86.95; ESIMS m/z 409.11 ([M+H]⁺).

### Step 2. Preparation of (4-(perfluoroethoxy)phenyl)hydrazine hydrochloride-¹⁵N₂

To a dry 250 mL round bottomed flask equipped with a magnetic stir bar, nitrogen inlet, and reflux condenser were added a solution of 1-(diphenylmethylene)-2-(4-(perfluoroethoxy)-phenyl)hydrazine-¹⁵N₂ (8.98 g, 22.0 mmol) in EtOH (40 mL) followed by concentrated HCl (9 mL, about 108 mmol), and the reaction was warmed to 80° C. and stirred for 15 h. The reaction was cooled to room temperature and the resulting tan solids were collected by vacuum filtration and washed on the filter with multiple portions of CH₂Cl₂ to give a white solid (Crop 1). The filtrate was concentrated on a rotary evaporator and the residue was slurried in CH₂Cl₂. The resulting solids were collected by vacuum filtration and washed as described above to give a white solid (Crop 2). Crops 1 and 2 were dried under vacuum and combined to give the title compound as a white solid (3.19 g, 52%): mp 217-223° C.; ¹H NMR (400 MHz, DMSO-d₆) δ 10.34 (s, 3H), 8.54 (d, J=82.7 Hz, 1H), 7.29 (d, J=8.9 Hz, 2H), 7.07 (dd, J=8.9, 1.4 Hz, 2H); ¹⁹F NMR (376 MHz, DMSO-d₆) δ −85.29, −87.00; ESIMS m/z 245.05 ([M+H]⁺).

### Example 4

### Preparation of methyl 4-(imino(methoxy)methyl)benzoate hydrochloride

[chemical structure]

To a magnetically stirred solution of methyl 4-cyanobenzoate (12.5 g, 78 mmol) in benzene (25 mL) and MeOH (7 mL) cooled to 0° C. was bubbled anhydrous HCl subsurface for 3 h. After storing in the refrigerator overnight, a heavy white precipitate formed. The solid was filtered through a fritted glass funnel and washed with diethyl ether to furnish the title compound as a white solid (17.5 g, 96%): mp 209-210° C.; ¹H NMR (400 MHz, CDCl₃) δ 13.05 (br s, 1H), 12.32 (br s, 1H), 8.48 (m, 2H), 8.22 (m, 2H), 4.60 (s, 3H), 3.97 (s, 3H); ¹³C NMR (101 MHz, DMSO-d₆) δ 166.98, 165.69, 138.38, 131.74, 129.00, 127.78, 52.29, 26.16; EIMS m/z 192 ([M⁺]).

### Example 5

### Preparation of methyl 4-(ethoxy(imino)methyl)benzoate hydrochloride

[chemical structure]

A 2 L, three-necked round bottomed flask equipped with a magnetic stir bar, a temperature probe, addition funnel and nitrogen inlet was charged with methyl 4-cyanobenzoate (100 g, 620 mmol). The methyl 4-cyanobenzoate was dissolved in EtOH (438 mL) and cooled in an ice bath to 0° C. Acetyl chloride (353 mL, 4960 mmol) was added dropwise into the stirring solution over a 2 h period during which time an exotherm from 0° C. to 21° C. was noted. The reaction flask was capped, sealed with Parafilm®, and allowed to stir at 23° C. for 18 h. the resulting white solid was collected by vacuum filtration and washed with EtOH. The filtrate was concentrated until it became turbid and was then cooled to 23° C. The resulting precipitate was collected by vacuum filtration, rinsed with EtOH, and the filtrate treated as described to give another crop. The solids were dried to give the title compound as a white solid (128 g, 85%): ¹H NMR (400 MHz, CDCl₃) δ 12.85 (br s, 1H), 12.20 (br s, 1H), 8.49 (m, 2H), 8.23 (m, 2H), 5.00 (q, 2H), 4.00 (s, 3H), 1.72 (t, 3H).

### Example 6

### Preparation of 4-(ethoxy(imino)methyl)benzoic acid hydrochloride and ethyl 4-(ethoxy(imino)methyl)benzoate hydrochloride

[chemical structure]

A 500 mL, three-necked flask, equipped with a magnetic stir bar, nitrogen inlet, addition funnel, and a temperature probe was charged with anhydrous EtOH (125 mL). The vessel was cooled to 5° C. and acetyl chloride (97 mL, 1332 mmol) was added at a rate that maintained the temperature range of 5° C. to about 10° C. When the addition was complete, 4-cyanobenzoic acid (25 g, 167 mmol) was added in portions over 15 min. No exotherm was noted during the addition of the solid. When the addition was complete, the white suspension was allowed to warm to 25° C. The reaction vessel was sealed with Parafilm® and stirred at 23° C. for 18 h. The white suspension was vacuum filtered and the solid was rinsed with EtOH and dried to constant mass, furnishing 4-(ethoxy(imino)methyl)benzoic acid hydrochloride as a white solid (25 g, 65%): ¹H NMR (400 MHz, DMSO-d₆) δ 12.44 (br s, 1H), 8.26 (m, 2H), 8.14 (m, 2H), 4.70 (q, 2H), 1.51 (t, 3H).

The filtrate was concentrated and treated with ether to give a white solid. The solid was collected by vacuum filtration and rinsed with ether to give ethyl 4-(ethoxy(imino)methyl)benzoate hydrochloride as a white solid (11 g, 25%): ¹H NMR (400 MHz, DMSO-d₆) δ 12.07 (br s, 1H), 8.24 (m, 2H), 8.15 (m, 2H), 4.66 (q, 3H), 4.37 (q, 3H), 1.49 (t, 3H), 1.35 (q, 3H).

### Example 7

### Preparation of ethyl 4-nitrobenzimidate hydrochloride

[chemical structure]

To a solution of 4-nitrobenzonitrile (27 g, 182 mmol) in EtOH (128 ml, 2187 mmol) under nitrogen was added acetyl chloride (104 ml, 1458 mmol) dropwise at 0° C. over 1 h, and the reaction was warmed to room temperature. The flask was sealed and the reaction was stirred for 56 h. The resulting precipitate (4-nitrobenzamide) was collected by filtration, and the filtrate was treated with diethyl ether. The resulting precipitate was collected by filtration, washed with diethyl ether and air dried to give the title compound (26.7 g, 58%): mp 198-200° C.; ¹H NMR (400 MHz, DMSO-d₆) δ 8.37-8.30 (m, 2H), 8.21-8.13 (m, 2H), 7.35 (s, 1H), 7.22 (s, 1H), 7.09 (s, 1H), 4.34 (q, J=7.1 Hz, 2H), 1.32 (t, J=7.1 Hz, 3H); EIMS m/z 193.

### Example 8

### Preparation of ethyl 4-nitrobenzimidate hydrochloride-¹³C,¹⁵N

[chemical structure]

To a 100 mL round bottomed flask equipped with a magnetic stir bar, temperature probe, nitrogen inlet, and liquid addition funnel was added anhydrous EtOH (5 mL) and the EtOH was cooled to 0° C. and treated dropwise with acetyl chloride (8.37 g, 107 mmol). To the resulting solution of HCl in EtOH was added a solution of 4-nitrobenzonitrile-¹³C,¹⁵N (2.0 g, 13.3 mmol) in EtOH (10 mL) dropwise between 0° C. and 8° C. The reaction mixture was warmed to room temperature and the flask was sealed with stoppers and Parafilm® and the contents were stirred for 17 h. The resulting precipitate was collected by filtration and washed with diethyl ether and EtOH, and the filtrate was treated with diethyl ether. The resulting precipitate was collected by filtration, washed with diethyl ether and air dried (repeated 2×) to give the title compound as a pale-yellow solid (2.20 g, 71%): mp 198-200° C.; ¹H NMR (400 MHz, DMSO-d₆) δ 12.87-11.58 (m, 1H), 8.43 (d, J=8.6 Hz, 2H), 8.33 (ddd, J=10.9, 5.0, 3.2 Hz, 2H), 4.65 (qd, J=6.9, 2.0 Hz, 2H), 1.49 (t, J=7.0 Hz, 3H).

### Example 9

### Preparation of methyl 4-(1-(4-(trifluoromethoxy)phenyl)-1H-1,2,4-triazol-3-yl)benzoate

[chemical structure]

To a magnetically stirred solution of methyl 4-(imino(methoxy)methyl)benzoate hydrochloride (1.15 g, 5.00 mmol) in anhydrous pyridine (5 mL) cooled by an ice bath was added (4-(trifluoromethoxy)phenyl)hydrazine hydrochloride (1.14 g, 5.00 mmol) in several portions. After warming to room temperature overnight (18 h), the yellow reaction mixture was diluted with water (25 mL) and washed with CH₂Cl₂ (2×50 mL). The combined organic layers were washed with brine, dried over MgSO₄, filtered, and concentrated to give an orange-yellow solid (1.60 g). The solid was dissolved in formic acid (15 mL), warmed to reflux, and stirred at reflux for 8 h. The reaction mixture was cooled to room temperature, diluted with water, and washed with diethyl ether (2×50 mL). The combined diethyl ether washes were washed with water (3×50 mL), washed with brine (50 mL), dried over MgSO₄, filtered, and concentrated on a rotary evaporator to give the title compound (1.42 g, 78%) as a tan solid. A sample was purified for analytical characterization by flash column chromatography using 0-100% EtOAc/hexanes (v/v) as eluent to give the product as an off-white solid: mp 171-172° C.; ¹H NMR (400 MHz, CDCl₃) δ 8.60 (s, 1H), 8.27 (m, 2H), 8.15 (m, 2H), 7.81 (m, 2H), 7.40 (d, J=8.5 Hz, 2H), 3.95 (s, 3H); ¹³C NMR (101 MHz, CDCl₃) δ 166.76, 162.61, 148.55, 141.77, 135.41, 134.46, 131.03, 130.02, 126.46, 122.44, 121.66, 121.31, 119.10; ESIMS m/z 363 ([M⁺]).

### Example 10

### Preparation of 4-(1-(4-(perfluoroethoxy)phenyl)-1H-1,2,4-triazol-3-yl)benzoic acid

[chemical structure]

A 1 L, three-neck round bottomed flask, equipped with an overhead mechanical stirrer, temperature probe, and nitrogen inlet was charged with 4-(ethoxy(imino)methyl)benzoic acid hydrochloride (25 g, 109 mmol) and pyridine (200 mL). The white suspension was cooled to 5° C. in an ice bath and (4-(perfluoroethoxy)phenyl)hydrazine hydrochloride (30.9 g, 109 mmol) was added in portions. The white suspension turned yellow and the temperature rose to 5.7° C. The ice bath was removed and the reaction mixture was allowed to warm slowly. At about 12° C., the contents of the flask became too thick to Air. Additional pyridine (20 mL) was added and warming continued for 20 min. The reaction mixture was poured into water (400 mL) resulting in precipitation of a flocculent solid. The mixture was extracted with CH₂Cl₂ (1×400 mL) and the phases were separated. The residual solid floating on top of the aqueous layer was collected by vacuum filtration and washed with CH₂Cl₂, and the aqueous filtrate was extracted with CH₂Cl₂ (2×200 mL). The organic extracts and washes were combined, washed with water (2×500 mL), and the water washes were back-extracted with CH₂Cl₂ until the aqueous layer was colorless (1×200 mL). The organic extracts were concentrated under reduced pressure to yield a dark, red oil (17.2 g). The previously isolated solid was suspended in CH₂Cl₂ (500 mL), stirred for 5 min, collected by filtration, rinsed on the filter with CH₂Cl₂, and dried under vacuum at 50° C. to give a bright yellow solid (20 g).

A 500 mL, three-necked, round bottomed flask, fitted with a magnetic stir bar, temperature probe, and nitrogen inlet, was charged with the dark red oil (17.2 g) and the bright yellow solid (20 g) isolated above. Formic acid (200 mL) was added and the mixture was heated to 100° C. and stirred for 16 h. The heat was removed and the mixture was allowed to cool. The mixture was cooled to 23° C. (precipitate forms at about 90° C.) and water (200 mL) was added. The mixture was stirred for 1 h and the solid was collected by vacuum filtration, washed with water, air dried, and then dried under vacuum at 50° C. for 2 days (d) to furnish the title compound as a light tan solid (22.3 g, 51%): ¹H NMR (400 MHz, DMSO-d₆) δ 13.13 (s, 1H), 9.48 (s, 1H), 8.23 (m, 2H), 8.10 (m, 4H), 7.64 (m, 2H).

### Example 11

### Preparation of 3-(4-nitrophenyl)-1-(4-(perfluoroethoxy)phenyl)-1H-1,2,4-triazole

[chemical structure]

To a stirred solution of ethyl 4-nitrobenzimidate hydrochloride (3 g, 13 mmol) in pyridine (13 mL) at 0° C. was added (4-(perfluoroethoxy)phenyl)hydrazine hydrochloride (3.62 g, 13.0 mmol) in three portions. The reaction mixture was warmed to room temperature and stirred for 2 h and was diluted with water and CH₂Cl₂. The phases were separated and the aqueous layer was extracted with CH₂Cl₂ (3×20 mL), and the combined organic fractions were washed with water (30 mL), dried over MgSO₄, and filtered. Concentration of the filtrate afforded a sticky red solid: ¹H NMR (400 MHz, CDCl₃) δ 8.34-8.23 (m, 2H), 8.00-7.90 (m, 2H), 7.20-7.07 (m, 4H), 6.33 (s 1H), 4.66 (s, 2H); ESIMS m/z 390 ([M]⁺).

The solid was added to formic acid (30 mL) and the reaction was heated to 100° C. and stirred for 18 h. The reaction mixture was cooled to room temperature and added to cold water. The resulting precipitate was collected by vacuum filtration, washed with water, and dried under vacuum to give the title compound as a light pink solid (4.99 g, 96%): mp 132-135° C.; ¹H NMR (400 MHz, CDCl₃) δ 8.68 (s, 1H), 8.43-8.31 (m, 4H), 7.89-7.80 (m, 2H), 7.49-7.38 (m, 2H); ESIMS m/z 400 ([M]⁺).

### Example 12

### Preparation of 3-(4-nitrophenyl)-1-(4-(perfluoroethoxy)phenyl)-1H-1,2,4-triazole-1,2,4-¹⁵N₃-3-¹³C

[chemical structure]

To a stirred solution of ethyl 4-nitrobenzimidate hydrochloride-¹³C-¹⁵N (2.13 g, 9.18 mmol) in pyridine (10 mL) at 0° C. was added (4(perfluoroethoxy)phenyl)hydrazine hydrochloride-¹⁵N₂ (2.57 g, 9.18 mmol) in portions. The reaction mixture was warmed to room temperature and stirred for 3 h and was diluted with water (20 mL) and CH₂Cl₂ (20 mL). The phases were separated and the aqueous layer was extracted with CH₂Cl₂ (3×20 mL), and the combined organic fractions were washed with water (2×40 mL), dried over MgSO₄, and filtered. Concentration of the filtrate afforded a sticky red solid.

The solid was dissolved in formic acid (20 mL) and the reaction was heated to 100° C. and stirred for 16 h. The reaction mixture was cooled to 28° C. and added to cold water (50 mL). The resulting precipitate was collected by vacuum filtration, washed with water, and dried. The crude product was purified by flash column chromatography using 0-60% EtOAc/hexanes (v/v) to give the title compound as a light tan solid (1.1 g, 30%): mp 133-136° C.; ¹H NMR (400 MHz, DMSO-d₆) δ 9.51 (td, J=11.9, 8.9 Hz, 1H), 8.34 (ddd, J=9.0, 6.6, 5.3 Hz, 4H), 8.15-8.06 (m, 2H), 7.63 (d, J=8.9 Hz, 2H); ESIMS m/z 405 ([M+H]⁺).

### Example 13

### Preparation of 4-(1-(4-(trifluoromethoxy)phenyl)-1H-1,2,4-triazol-3-yl)benzoic acid

[chemical structure]

To a solution of methyl 4-(1-(4-(trifluoromethoxy)phenyl)-1H-1,2,4-triazol-3-yl)benzoate (0.332 g, 0.914 mmol) in THF (6 mL) and water (3 mL) was added LiOH (0.066 g, 2.74 mmol), and the solution immediately turned from yellow to orange-red. The reaction was stirred vigorously at room temperature for 16 h. The solution was acidified to pH 2 and diluted with water and CH₂Cl₂. The phases were separated and the aqueous layer was extracted with EtOAc (3×10 mL) and the combined organic fractions were washed with water (10 mL), washed with brine (10 mL), dried over MgSO₄, filtered, and concentrated to give the title compound as a tan solid (0.29 g, 91%): mp 228-233° C.; ¹H NMR (400 MHz, DMSO-d₆) δ 10.55-10.24 (m, 1H), 9.46 (s, 1H), 8.23 (d, J=8.0 Hz, 2H), 8.09 (d, J=7.9 Hz, 4H), 7.64 (d, J=8.5 Hz, 2H); ESIMS m/z 350 ([M+H]⁺).

### Example 14

### Preparation of 4-(1-(4-(perfluoroethoxy)phenyl)-1H-1,2,4-triazol-3-yl)benzoic acid (Alternative to Example 10)

[chemical structure]

In a 250 mL round bottomed flask equipped with an overhead stirrer, T-type thermocouple, and nitrogen inlet were added methyl 4-(1-(4-(perfluoroethoxy)phenyl)-1H-1,2,4-triazol-3-yl)benzoate (11.1 g, 26.9 mmol) and THF (100 mL). To this yellow suspension were added water (10 mL) and lithium hydroxide.monohydrate (LiOH.H₂O; 3.4 g, 81 mmol). The reaction was stirred at 23° C. for 39 h during which time it became a yellow solution. The solution was warmed to 60° C. and stirred at 60° C. until complete by LC-MS. The reaction was cooled to 4° C. in an ice bath and water (100 mL) was added to give a light yellow solution. Concentrated HCl (8.0 g) was added (note: exothermic) resulting in a thick white precipitate. The white suspension was stirred at 5° C. for 30 min and then the solid was collected by vacuum filtration. The filter cake was washed with water (2×25 mL), air dried for 3 h, and dried under vacuum (700 mm Hg) at 50° C. for 16 h to give the title compound as a white solid (10.3 g, 96%): mp 227-229° C.; ¹H NMR (400 MHz, CDCl₃) δ 8.65 (s, 1H), 8.32 (d, J=8.4 Hz, 2H), 8.23 (d, J=8.4 Hz, 2H), 7.84 (d, J=8.9 Hz, 1H), 7.42 (d, J=8.9 Hz, 2H).

### Example 15

### Preparation of 4-(1-(4-(perfluoroethoxy)phenyl)-1H-1,2,4-triazol-(1,2,4-¹⁵N₃-3-¹³C)-3-yl)aniline

[chemical structure]

To a magnetically stirred solution of 3-(4-nitrophenyl)-1-(4-(perfluoroethoxy)phenyl)-1H-1,2,4-triazole-1,2,4-¹⁵N₃-3-¹³C (1.1 g, 2.72 mmol) in EtOAc (40 mL) was added palladium on carbon (10% wt., 110 mg), and the reaction flask was evacuated and purged with hydrogen (Repeated 4×). The reaction mixture was placed under an hydrogen atmosphere (balloon) and stirred at room temperature for 2.5 h. The mixture was filtered through Celite® and concentrated on a rotary evaporator to give the title compound as a white solid (0.92 g, 90%): mp 177-180° C.; ¹H NMR (400 MHz, DMSO-d₆) δ 9.28 (td, J=11.8, 8.9 Hz, 1H), 8.11-8.01 (m, 2H), 7.86-7.75 (m, 2H), 7.59 (d, J=8.9 Hz, 2H), 6.66 (d, J=8.3 Hz, 2H), 5.51 (s, 2H); ESIMS m/z 375 ([M+H]⁺).

### Example 16

### Preparation of (2S,3R,4R,5S,6S)-3,5-dimethoxy-6-methyl-4-propoxytetrahydro-2H-pyran-2-yl (4-(1-(4-(perfluoroethoxy)phenyl)-1H-1,2,4-triazol-(1,2,4-¹⁵N₃-3-¹³C)3-yl)phenyl)carbamate

[chemical structure]

To a magnetically stirred solution of 4-(1-(4-(perfluoroethoxy)phenyl)-1H-1,2,4-triazol-(1,2,4-¹⁵N₃-3-¹³C)-3-yl)aniline (0.400 g, 1.07 mmol) in anhydrous MeCN (10 mL) was added 4-nitrophenylchloroformate (0.215 g, 1.07 mmol) and within min a white precipitate formed. The thick mixture was stirred for 1 h, at which time HPLC-MS analysis indicated full conversion of the aniline starting material to the desired carbamate (ESIMS m/z 540 ([M+H]⁺). Potassium phosphate (K₃PO₄, 0.045 g, 0.21 mmol) was added to the mixture followed by a solution of (2R,3R,4R,5S,6S)-3,5-dimethoxy-6-methyl-4-propoxytetrahydro-2H-pyran-2-ol (0.275 g, 1.18 mmol), prepared as described by Crouse et. al. in WO 2009102736 A1, in CH₃CN (2 mL) and DIPEA (0.276 g, 2.14 mmol), at which point the yellow-orange mixture became easier to stir. The reaction was stirred for 16 h at room temperature, concentrated on a rotary evaporator, and the yellow-brown residue was partitioned between EtOAc (20 mL) and water (20 mL). The phases were separated and the organic phase was washed with water (2×20 mL), washed with brine (20 mL), dried over MgSO₄, filtered, and the filtrate concentrated to give the crude product as a yellow-brown foam. The foam was purified by reverse phase column chromatography (C18) using 5-95% MeCN/water (0.1% trifluoroacetic acid (TFA) as eluent to give an oily, yellow foam (isolated 115 mg). The residual foam that could not be scraped from the flask (180 mg) was treated with heptane (3 mL) and warmed to 80° C. After stirring for ˜5 min the foam had transformed into a solid. The solid was collected by vacuum filtration and dried to give the title compound as a tan solid (0.261 g total, 38%): mp 142-146° C.; ¹H NMR (400 MHz, CDCl₃) δ 8.59 (td, J=11.6, 8.7 Hz, 1H), 8.20-8.13 (m, 2H), 7.86-7.77 (m, 2H), 7.54 (d, J=8.0 Hz, 2H), 7.40 (d, J=8.9 Hz, 2H), 6.85 (s, 1H), 6.20 (d, J=1.8 Hz, 1H), 3.77-3.49 (m, 11H), 3.21 (t, J=9.4 Hz, 1H), 1.68 (dd, J=14.1, 7.3 Hz, 2H), 1.34 (t, J=8.7 Hz, 3H), 0.98 (dd, J=9.5, 5.3 Hz, 3H); ESIMS m/z 635 ([M+H]⁺).

What is claimed is:

1. A process comprising: [chemical structure]
(1a) reacting a haloalkoxyarylhalide of Formula 1 with a hydrazone of Formula 1.1 in the presence of a palladium catalyst complex comprising palladium and a ligand to produce an intermediate haloalkoxyarylhydrazone of Formula 1.2; followed by
(1b) hydrolyzing said intermediate haloalkoxyarylhydrazone of Formula 1.2 to produce a haloalkoxyarylhydrazine of Formula 1.3;
wherein R is a (C₁-C₆)haloalkoxy; X is F, Cl, Br, or I; n=0, 1, or 2; wherein R₁ and R₂ are independently aryl, heteroaryl, substituted aryl, substituted heteroaryl, or tertiary alkyl, wherein the substituents do not adversely affect the reaction.

2. A process according to claim 1 further comprising: [chemical structure]
(2a) reacting said haloalkoxyarylhydrazine of Formula 1.3 with an arylalkoxyimidate of Formula 2.2 to produce an intermediate iminohydrazine of Formula 3.1; followed by
(2b) cyclizing said intermediate iminohydrazine of Formula 3.1 using a formate source to produce a 1,3-diaryltriazole of Formula 3.2;
wherein R₄ is NO₂, C(═O)OH, or C(═O)O(C₁-C₆)alkyl; and R₃ is (C₁-C₆)alkyl.

3. A process according to claim 1 wherein (1 a) R is trifluoromethoxy or pentafluoroethoxy.

4. A process according to claim 1 wherein (1a) X is Br.

5. A process according to claim 1 wherein (1a) R₁ and R₂ are phenyl.

6. A process according to claim 1 wherein (1a) palladium catalyst complex is tetrakis(triphenyl-phosphine)palladium(0), tris(dibenzylideneacetone)dipalladium(0) (Pd₂(dba)₃), or is prepared in situ by contacting palladium (II) acetate (Pd(OAc)₂); palladium (II) chloride (PdCl₂), sodium tetrachloropalladate (Na₂PdCl₄); bis(dibenzylideneacetone) palladium(II) (Pd(dba)₂), with a ligand, selected from triphenylphosphine (PPh₃), 1,1′-bis(diphenylphosphino)ferrocene (dppf), dibenzylideneacetone (dba); 2′-bis(diphenylphosphino)-1,1′-binaphthyl (BINAP); or 2-dicyclohexylphosphino-2′-methylbiphenyl (MePhos).

7. A process according to claim 1 wherein (1a) a molar ratio of the haloalkoxyarylhalide of Formula 1 and a hydrazone of Formula 1.1 is from about 1.5:1 to about 1:1.5.

8. A process according to claim 1 wherein (1a) the reaction of the haloalkoxyarylhalide of Formula 1 with the hydrazine of Formula 1.1 is conducted in the presence of sodium tert-butoxide (NaO^{t}Bu), potassium tert-butoxide (KO^{t}Bu), lithium hexamethyldisilazide (LiHMDS), sodium carbonate (Na₂CO₃), potassium carbonate (K₂CO₃), cesium carbonate (Cs₂CO₃), tripotassium phosphate (K₃PO₄), or mixtures thereof.

9. A process according to claim 1 wherein (1a) the pH is from about 12 to about 14.

10. A process according to claim 1 wherein (1a) the temperature from about 95-100° C.

11. A process according to claim 1 wherein (1b) is conducted in methanol or ethanol.

12. A process according to claim 1 wherein (1b) the pH is from about −1 to about 1.

13. A process according to claim 2 wherein (2a) is conducted in pyridine, lutidine, or mixtures thereof.

14. A process according to claim 2 wherein (2b) is conducted at a temperature from about 95 to about 100° C.

15. A process according to claim 2 wherein (2b) formate source is formic acid.

16. A process according to claim 2 wherein: R is pentafluoroethoxy; and R₄ is NO₂, C(═O)OH, C(═O)OCH₃, or C(═O)OCH₂CH₃.

17. Intermediate haloalkoxyarylhydrazones of Formula 1.2 having the following structures [chemical structure]
